(19) United States
(12) Patent Application Publication
(10) Pub. No.: US 2004/0034413 A1
(43) Pub. Date: Feb. 19, 2004

(54) Hydrogel corneal inlay

(76) Inventor: James M. Christensen, Glendora, CA (US)

Correspondence Address: Michael J. Ram, KOPPEL, JACOBS, PATRICK & HEYBL, 555 St. Charles Drive, Suite 107, Thousand Oaks, CA, 91360, US

(21) Appl. No.: 10/217,754

(22) Filed: Aug. 13, 2002

(51) Int. Cl. A61F 2/14

(52) U.S. Cl. 623/5.11

(57) ABSTRACT

A hydrogel corneal inlay for implantation under a lamellar dissection of the cornea to modify the anterior corneal curvature, thereby altering the refractive power of the eye, in the treatment of hyperopia, myopia, astigmatism, and presbyopia. The inlay front and rear surfaces are the same configuration so the function of the inlay will not depend on which surface of the inlay is placed against the stroma.

### FIELD OF THE INVENTION

This invention relates to a hydrogel inlay designed to be surgically inserted into the stromal layers of the eye thereby altering the corneal curvature of the eye to correct refractive errors.

### BACKGROUND OF THE INVENTION

It is well known in the prior art that altering the curvature of the cornea can change the refractive power of the eye. Excimer laser refractive surgery is based on the removal of a thin amount of tissue from the cornea to alter the corneal curvature thereby correcting a patient's vision. This process works best in the treatment of myopia where corneal tissue in the shape of a lens is removed from the central portion of the cornea thereby flattening the corneal curvature. This results in a cornea with an increased radius of curvature. A less predictable correction is obtained for hyperopia where a thin amount of tissue in the form of a washer is removed from the periphery of the cornea thereby steepening the corneal curvature. This results in a cornea with a smaller radius of curvature having depressions where the outer edge of the washer is ablated into the cornea. It is this area of depression that the cornea fills in over time thereby resulting in a loss of the hyperopic correction.

Various styles of implants have been placed in the corneal stroma since 1964, initially to control corneal edema, but more recently to correct refractive errors. U.S. Pat. No. 4,607,617 to Choyce describes a corneal inlay constructed of polysulfone plastic which had a high refractive index (typically 1.633). This inlay had an appearance resembling a conventional contact lens (bi-meniscus in shape) to match the curvature of the cornea and was designed to be inserted into a pocket formed between the layers of the stromal tissue. It had a thickness in the range of 0.1 mm to 0.4 mm. In animals and humans this inlay, and other similar inlays, which were formed from non-permeable plastic, functioned poorly causing anterior corneal necrosis due to a lack of permeability to fluids and nutrients.

U.S. Pat. No. 5,336,261 to Barrett et. al. describes a variety of very small diameter lenses having an inherent optical power such that when placed in the cornea they have an index of refraction greater that the corneal tissue. These lenses were substantially smaller (approximately 70%) than the patient's optical zone in bright light conditions which usually ranges from 2 mm to 3 mm in diameter. They were formed from low to high refractive index materials. The diameter of the lens was reduced in an attempt to increase fluid and nutrient transport around the implant. These lenses had to be implanted deep in the corneal stroma to obtain sufficient transport to prevent anterior corneal necrosis. However, they do not prevent the long term formation of corneal opacities adjacent to the anterior surface of the lens due to inadequate fluid and nutrient transport to this region. Since the lenses are smaller than the patient's optical zone, multiple images are focused onto the cornea. These images can cause glare and result in poor vision in low light level conditions in many patients.

Hydrogel inlays have been more successful than other types of inlays for corneal implant. The first hydrogel inlays were inserted into the cornea by Mester in 1976. Since then a variety of hydrogel formulations have been tried with various degrees of success. Hydrogel inlays allow the transport of fluids and nutrients across the inlay in proportion to the water content and thickness of the hydrogel. The most successful inlays were formed from high water content hydrogels typically around 70% water content. However, these formulations of hydrogel have an index of refraction that approaches that of the corneal stroma (typically 1.376) and therefore have no inherent optical power when placed in the stroma. They cannot provide a refractive change to the eye without altering the anterior curvature of the cornea.

Various designs of bi-meniscus solid and washer shaped hydrogel corneal inlays have been tried in animals. These types of hydrogel inlays have been inserted into the stroma predominantly by making a tunnel and/or pocket within the cornea through a small incision which leaves Bowman's membrane intact. This does not relieve the natural tension of this membrane and therefore these inlays do not significantly alter the anterior curvature of the cornea. Since the amount of Bowman's membrane that is cut during the insertion of these inlays is variable, the refractive correction achieved is not accurately predictable and corneal irregularities from unequal residual tension in Bowman's membrane can be obtained.

With the use of a microkeratome, surgeons can produce a flap in the anterior surface of the cornea which can then be folded back off the central portion of the cornea. This allows the surgeon to use an excimer laser to ablate a change into the contour of the underlying stromal tissues. Since Bowman's membrane has been cut in producing the flap, there is no residual stress left in this membrane. When the flap is laid back over the cornea it will conform to the newly created contour in the stroma and provide a refractive change to the eye. This procedure works well for myopic eyes where the curvature of the cornea needs to be flattened and material is removed predominantly from the central portion of the cornea.

Recently, U.S. Pat. Nos. 6,361,560 and 6,102,946 to Nigam describe high water content hydrogel corneal inlays that are bi-meniscus in shape so that they conform to the curvature of the cornea. They are designed to be implanted under a corneal flap formed by a microkeratome. These inlays have a center thickness of no greater than about 50 microns so that adequate fluid and nutrient transport is provided to the corneal flap anterior to the inlay. When the corneal flap is laid back over these inlays, it will conform to the newly created contour thereby causing a change to the anterior corneal curvature.

The hydrogel corneal inlays described by Nigam have anterior surface and posterior surface curves with a different radius of curvature. This results in a bi-meniscus (contact lens style) inlay where the radius of curvature of the anterior and posterior surfaces can be altered to obtain different thicknesses in the central or peripheral portion of the inlay. One of the problems with this style lens, which is well know by contact lens wearers, is that it is easy to invert the lens so that the anterior and posterior surfaces of the lens are reversed. This causes discomfort in the case of contact lens wearers. Clinical usage of the Nigam style corneal inlays has shown that it is very difficult to tell if the corneal inlay has been inverted. Clinical results have shown that a different corneal correction is achieved if the inlay is insert in its inverted condition. This shift in refractive power is not acceptable to the patient.

Thus there is still a need for a corneal inlay that will allow for the transport of fluids and nutrients across the implant, that is thin enough so that it can be inserted under a corneal flap, and does not provide a refractive shift if it is implanted in an inverted configuration.

### SUMMARY OF THE INVENTION

The present invention provides an optically clear, biocompatible, flexible, high water content hydrogel corneal inlay that is designed to be implanted under a lamellar dissection made in the cornea (such as a corneal flap) to relieve tension in Bowman's membrane so that when the lamellar flap is laid back over the inlay it will conform to the contour created by the inlay. The corneal inlay is symmetrical in shape, formed by identical curvature anterior and posterior surfaces, so that implantation of the inlay into the stromal tissues of the eye does not have a preferred anterior to posterior orientation.

For correction of hyperopia the inlay is preferably circular, biconvex in shape with an outer diameter greater than the size of the pupil in bright light conditions. The central thickness is approximately 50 microns. Refractive change is achieved by varying, by the same amount, the radius of curvature for the anterior and posterior surfaces of the inlay which results in a change in the outer diameter and central thickness of the inlay. The radius of curvature for the anterior (Rₐ) and posterior (Rₚ) surfaces are substantially the same.

For correction of myopia the inlay is preferably circular biconcave in shape with an outer diameter greater than the size of the pupil in bright light conditions. The peripheral thickness is approximately 50 microns. The central portion of the inlay can be either very thin to obtain a solid style inlay or not present to obtain a washer style inlay. Refractive change is preferably achieved by varying the radius of curvature equally for the anterior (Rₐ) and posterior (Rₚ) surfaces of the inlay which results in a change in the outer diameter and central thickness of the inlay.

For correction of astigmatism a cylindrical component is added to the inlay. The resultant inlay is preferably elliptical in shape with a thickness of approximately 50 microns. The cylindrical refractive component is achieved by varying the radius of curvature for the X axis and Y axis of the inlay which results in a change to the dimensions of the elliptical inlay. The resultant curvatures are identical for both the anterior and posterior surfaces of the inlay.

For correction of presbyopia a multifocal component is added to the inlay. The resultant inlay is preferably circular in shape with a thickness of approximately 50 microns. The multifocal refractive component is achieved by adding an additional radius of curvature equally to the anterior and posterior surfaces of the inlay in the central 1.5 mm to 3 mm central portion of the inlay.

These and other aspects of the present invention will become apparent by reference to the specifications below and the appended claims.

### BRIEF DESCRIPTION OF THE DRAWINGS

A better understanding of the invention can be obtained from the specifications set forth below, when considered in conjunction with the appended drawings, in which:

FIG. 1 is a schematic representation of a cornea showing a lamellar dissection to produce a corneal flap;

FIG. 2 is a schematic representation of a cornea in which an inlay has been implanted for hyperopic correction;

FIG. 3 is a schematic representation of a cornea in which an inlay has been implanted for myopic correction;

FIG. 4 is a plan view of a solid corneal inlay for correcting hyperopia;

FIG. 5 is a cross-sectional view taken along line 5-5 of FIG. 4.

FIG. 6 is a plan view of a solid corneal inlay for correcting myopia;

FIG. 7 is a cross-sectional view taken along line 7-7 of FIG. 6.

FIG. 8 is a plan view of a washer shaped corneal inlay for correcting myopia;

FIG. 9 is a cross-sectional view taken along line 9-9 of FIG. 8 FIG. 10 is a plan view of a hyperopic, elliptical inlay for correcting astigmatism where the two axes have different curvatures;

FIGS. 11 and 12 are cross-sectional views taken along lines 11-11 and 12-12 of FIG. 10.

FIG. 13 is a plan view of a myopic, elliptical inlay for correcting astigmatism where the two axes have different curvatures;

FIGS. 14 and 15 are cross-sectional views taken along lines 14-14 and 15-15 of FIG. 13;

FIG. 16 is a plan view of a corneal inlay for correcting presbyopia with an additional refractive zone in the central portion of the inlay;

FIG. 17 is a cross-section view taken along line 17-17 of FIG. 16.

### DETAILED DESCRIPTION OF THE INVENTION

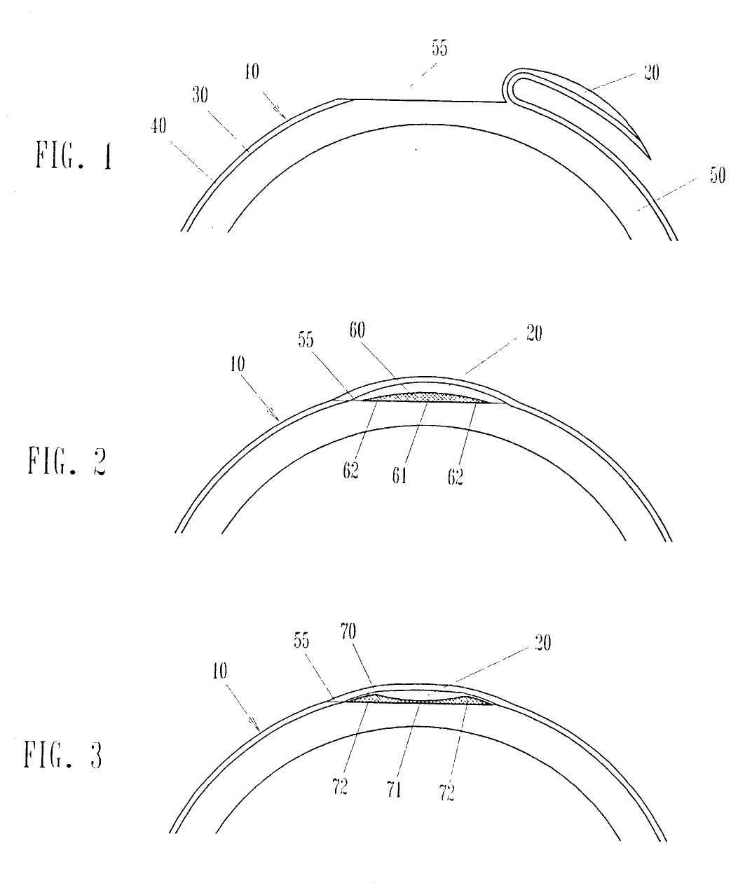

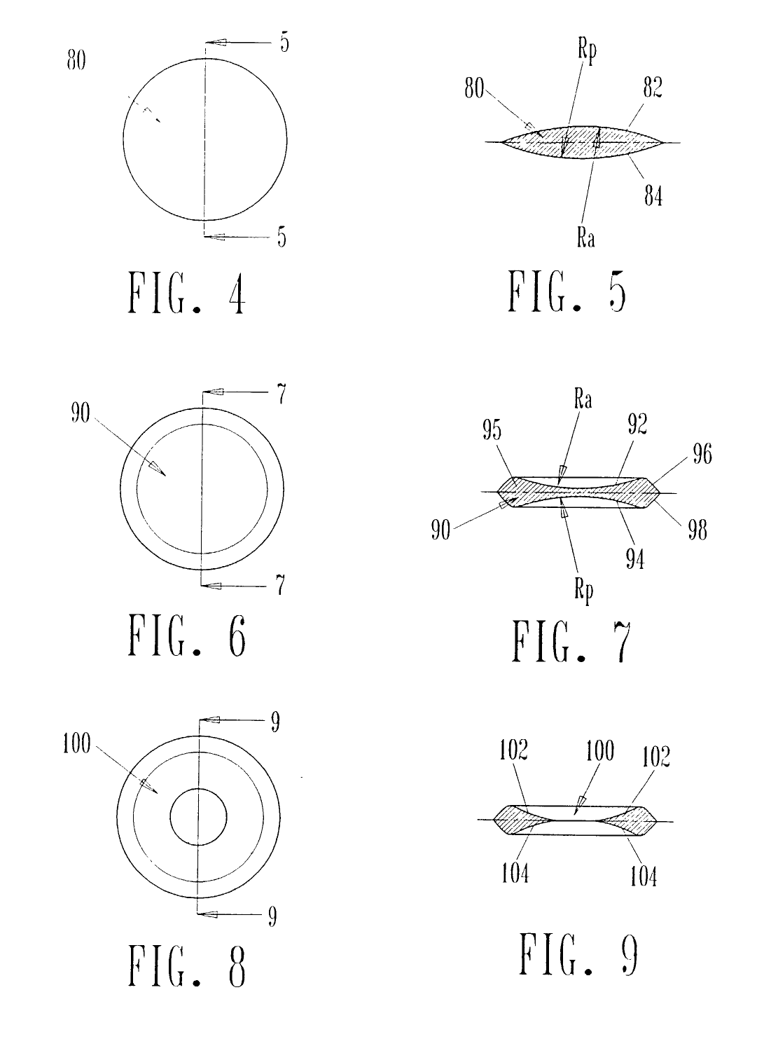

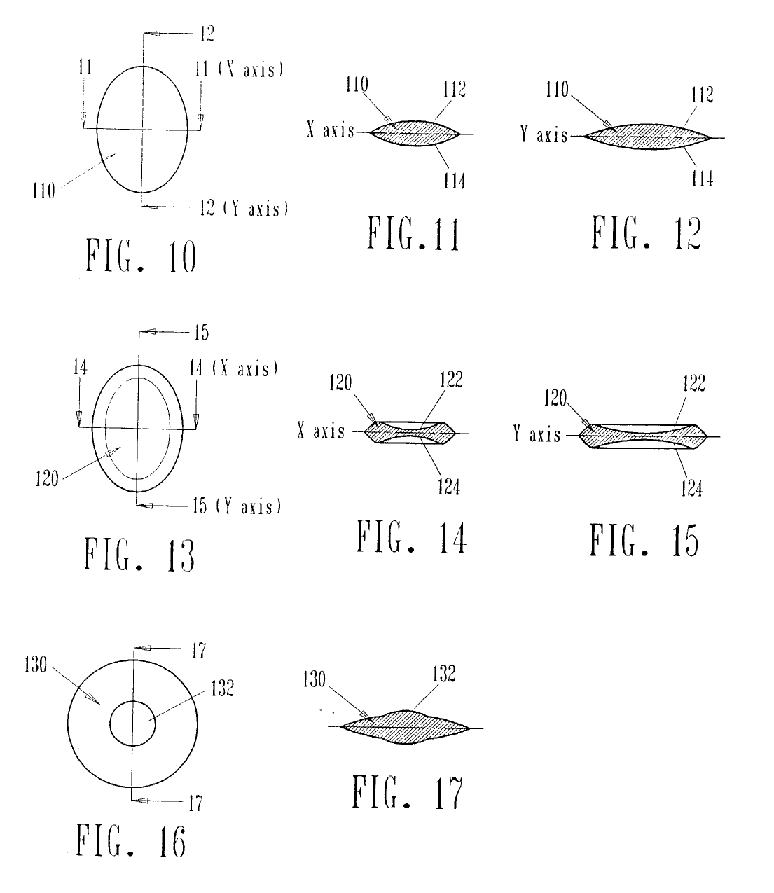

FIG. 1 shows the cornea 10 of an eye after it has been prepared for an inlay by lamellar dissection. A corneal flap 20 has been partially cut from the anterior surface of the cornea 10 preferably by using a microkeratome. The corneal flap 20 has been laid back off the central portion of the cornea thereby exposing a stromal surface 55 underneath it where the inlay will be placed. Bowman's membrane 30, which is comprised of a single layer of cells situated between the epithelial layer 50 and the stromal layer 40 of the cornea has been cut through which relieves the natural tension in this membrane. This will allow the corneal flap to conform to the contour of the corneal inlay when the corneal flap is replaced back over the cornea in its normal position.

Referring to FIGS. 2 and 3, there are seen corneal inlays 60 and 70 implanted into the cornea. These corneal inlays were placed onto the stromal surface 55 and the corneal flap 20 was laid back over the inlay. Shown in FIG. 2 is a corneal inlay 60 that has a thicker central portion 61 and a thinner peripheral portion 62. This causes the overlying corneal flap 20 to bulge further forward creating a steeper corneal curvature (smaller radius of curvature). This style of corneal inlay is suitable for correction of hyperopia by adding to the positive Diopter power of the cornea.

Shown in FIG. 3 is a corneal inlay 70 that has a thinner central portion 71 and a thicker peripheral portion 72. This causes the overlying corneal flap 20 to depress in the central portion somewhat thereby creating a flatter corneal curvature (larger radius of curvature). This style of corneal inlay is suitable for correction of myopia by reducing the positive Diopter power of the cornea.

The corneal inlays 60 and 70 discussed above are preferably formed from an optically clear, biocompatible, high water content hydrogel having a water content greater than 70%. Such high water content hydrogels have been shown to provide adequate fluid and nutrient transport when used in corneal inlays measuring over 250 microns in thickness. The preferred corneal inlays have a thickness of about 50 microns in order to provide adequate fluid and nutrient transport to the corneal flap, to be able to be handled, and to prevent excessive deformation of the corneal flap. However, the maximum thickness should be no greater than about 100 microns to avoid excessive corneal flap deformation.

High water content hydrogels having a water content greater than 70% have an index of refraction that approaches that of corneal tissue. Inlays made from these hydrogels provide a refractive change to the eye by altering the anterior curvature of the cornea. The change is not due to the optical characteristics of the inlay composition. These hydrogels are very flexible and compliant, especially when used in a corneal inlay having a thickness of approximately 50 microns. When placed on the stromal tissue surface 55 of the cornea, the corneal inlay will take on the curvature of the stromal surface as shown by the corneal inlays 60 and 70 in FIGS. 2 and 3. In the process of the inlay deforming to match the contour of the stromal surface 55, the anterior surface of the inlay stretches and is placed in tension and the posterior surface, which contacts the stromal surface 55, is placed into compression. This process alters the thickness of the corneal inlay by a determinable amount based on the amount of tension and compression placed on the inlay. However, in order to prevent a change in anterior corneal curvature if the inlay is inverted (put in upside down), it is necessary that the inlay be symmetrical in design with both surfaces (anterior and posterior) being identical, so that there is no longer a distinction between anterior or posterior surface of the inlay prior to implantation. In contrast, a non-symmetrical inlay will have a different amount of tension and compression placed on the inlay depending on whether the inlay is implanted correctly, or inverted. This difference causes the inlay to have a different thickness and will therefore result in a different refractive correction to the patient dependent on which surface is anterior.

Referring to FIGS. 4 and 5, there is shown a preferred embodiment of a corneal inlay 80 suitable for the correction of hyperopia. The corneal inlay 80 is circular in- shape and biconvex in style. Both the first surface 82 and the second surface 84 of the inlay have the same radius of curvature (Rₐ and Rₚ) so that the corneal inlay is symmetrical—that is, the curvatures of the first surface (Rₐ) and second surface (Rₚ) are identical. This corneal inlay can be implanted into the cornea with either the first surface 82 or the second surface 84 facing anteriorly and the optical correction to the eye will be the same. The change in anterior corneal surface curvature caused by this inlay is determined by the central thickness and the diameter of the inlay. Preferably, corneal inlay 80 will have a central thickness of approximately 50 microns and a diameter which ranges from 3 mm to 6 mm. It is clear from FIG. 4 that the inlay is biconvex in shape before placement in the cornea. However, when compared with FIG. 2, once implanted, the hydrogel inlay conforms to the stromal surface 55, as indicated above, which causes the surface of the inlay placed onto the stromal surface to take on the shape of the stromal surface.

Referring to FIGS. 6 and 7, there is shown a corneal inlay 90 suitable for the correction of myopia. The corneal inlay 90 is circular in shape and its central portion is biconcave in style. Both the first surface 92 and the second surface 94 in the central portion of the inlay have an identical radius of curvature (Rₐ and Rₚ). The corneal inlay 90 has a transition zone 95 formed by a first peripheral surface 96 and a second peripheral surface 98 which are also identical. The resultant corneal inlay 90 is completely symmetrical and therefore can be implanted into the cornea with either the first central surface 92 or the second central surface 94 facing anteriorly. The change in anterior corneal surface curvature caused by this inlay is determined by the peripheral thickness, the central thickness and the diameter of the inlay. Preferably, corneal inlay 90 will have a peripheral thickness at the transition zone of approximately 50 microns and a diameter which ranges from 3 mm to 6 mm. In comparing FIG. 5 with FIG. 3, it will also be noticed, that when the inlay is placed on the stromal surface, it will conform to the stromal surface and the inlay's shape will appear to be different.

Alternatively, instead of using a solid corneal inlay 90 as shown in FIGS. 6 and 7 for correcting myopia, a washer style corneal inlay as shown in FIGS. 8 and 9 could be used. This washer style corneal inlay 100 is formed substantially the same as the solid style corneal inlay shown in FIGS. 6 and 7 except that radius of curvature of the surfaces 102 and 104 forming the concave portion of the inlay is small enough so that part of the central portion of the inlay becomes a void, thereby forming a washer or ring shape. The resultant corneal inlay 100 is symmetrical in shape.

Biconvex hyperopic corneal inlays can be formed with a cylindrical addition in one axis of the inlay in order to correct for astigmatism as shown in FIGS. 10, 11 and 12. The resultant corneal inlay 110 is elliptical in shape with one axis (Y) longer that the axis (X) perpendicular to it. The thickness of the center of the corneal inlay 110 shown in the X and Y sectional views of FIGS. 11 and 12 taken along lines 11-11 and 12-12 of FIG. 10 is the same. The cylindrical power addition for the correction of astigmatism is obtained by varying the radius of curvature, and thereby the dimensions of the ellipse, in each of these axes. For the biconvex corneal inlay 110 shown in FIG. 10, there is a greater positive diopter power in the X axis, which has a smaller radius of curvature, compared to the Y axis which has a longer radius of curvature. The curvatures forming the first surface 112 of the corneal inlay 110 are the same as the curvatures forming the second surface 114 of the corneal inlay. Therefore, the curvatures are identical and the resultant corneal inlay is symmetrical.

Similarly, biconcave myopic corneal inlays can be formed with a cylindrical addition in one axis of the inlay in order to correct for astigmatism as shown in FIGS. 13, 14 and 15. The resultant corneal inlay 120 is elliptical in shape with one axis (Y) longer that the axis (X) perpendicular to it. The thickness of the center of the corneal inlay 120 shown in the X and Y sectional views of FIGS. 14 and 15, respectively, is the same. The cylindrical power addition for the correction of astigmatism is obtained by varying the radius of curvature, and thereby the dimensions of the ellipse, in each of these axes. For the biconcave corneal inlay 120 shown in FIG. 13, there will be a greater negative diopter power in the X axis, which has a smaller radius of curvature in its central portion, compared to the Y axis which has a longer radius of curvature. The result is a raised portion, or ring spaced inward of the periphery and outward from the center of the inlay. The curvatures forming the first surfaces 122 of the corneal inlay 120 are the same as the curvatures forming the second surfaces 124 of the corneal inlay. Therefore, the curvatures are identical and the resultant corneal inlay is symmetrical.

Referring to FIGS. 16 and 17, a corneal inlay 130 having a multifocal surface is shown. Such an inlay is suitable for the correction of presbyopia in a patient. The multifocal feature, which constitutes two additional biconvex portions centrally located on the first and second biconvex portions, is added to the corneal inlay 130. The curved surface 132 in the central 1.5 mm to 3 mm diameter, most preferably 2 mm diameter, portion of the inlay has a smaller radius of curvature resulting in what appears to be a hemispheric bulge on the implant surface. This provides a second refractive surface that is smaller that the patient's optical zone in bright light conditions. In addition to the base corneal inlay which may have no correction, or a correction for hyperopia or myopia, a cylindrical addition may be provided. Whatever changes are made to the first surface of the corneal inlay, the identical changes are made to the second surface of the corneal inlay. Thus both surfaces are identical and the inlay is symmetrical in shape.

Various modifications and alterations to the invention will now be perceived by those who have had the benefit of the applicant's teaching herein. Such alteration might be the addition of a cylinder component to the multifocal inlay shown in FIGS. 16 and 17. However, it will be understood that all such modifications and additions are deemed to be within the scope of the invention which is to be limited only by the claims appended hereto.

I claim:

1. A corneal inlay for providing a change to the anterior corneal curvature of a patient's eye when placed under a corneal flap comprising:
a) an optically clear structure comprised of a flexible, biocompatible material having an index of refraction substantially the same as corneal tissue,
b) the structure having a first surface and a second surface, the first surface and the second surface being joined at a periphery of the structure, the first and second surfaces having identical curvatures,
c) the structure being no greater than 100 microns at its thickest point.

2. The corneal inlay of claim 1 wherein the biocompatible material is a hydrogel.

3. The corneal inlay of claim 2 wherein the hydrogel contains at least about 70% water.

4. The corneal inlay of claim 1 wherein the structure is biconvex.

5. The corneal inlay of claim 4 wherein the periphery of the structure forms an ellipse.

6. The corneal inlay of claim 1 wherein a central portion thereof is biconcave.

7. The corneal inlay of claim 6 wherein the periphery of the structure forms an ellipse.

8. The corneal inlay of claim 6 wherein the thickest point comprises a raised circular portion spaced in from the periphery of the structure forming a ring.

9. The corneal inlay of claim 6 wherein a central portion of the structure is absent.

10. The corneal inlay of claim 4 wherein the first and second surfaces of the structure have a centrally located portion having identical curvatures with a smaller radius of curvature than the radius of curvature of the remainder of the first and second surface.

11. A method of treating hyperopia comprising forming a corneal flap, inserting an inlay between the corneal flap and the stroma and replacing the corneal flap with the inlay sealed there between, such that the contour of the anterior corneal surface is modified, the improvement comprising:
a) using an inlay which is biconvex in structure, optically clear and comprised of a flexible, biocompatible material having an index of refraction substantially the same as corneal tissue,
b) the inlay having a first surface and a second surface, the first surface and the second surface being joined at a periphery of the inlay, the first and second surfaces having identical curvatures,
c) the inlay being no greater than 100 microns at its thickest point.

12. A method of treating myopia comprising forming a corneal flap, inserting an inlay between the corneal flap and the stroma and replacing the corneal flap with the inlay sealed there between, such that the anterior contour of the corneal surface is modified, the improvement comprising:
a) using an inlay having an outer periphery, a raised transition zone spaced inward from the periphery and a biconcave central portion, the inlay being optically clear and comprised of a flexible, biocompatible material having an index of refraction substantially the same as corneal tissue,
b) the central portion of the inlay having a first surface and a second surface, the first surface and the second surface having identical curvatures,
c) the raised area being no greater than 100 microns at its thickest point.

13. A method of treating presbyopia comprising forming a corneal flap, inserting an inlay between the corneal flap and the stroma and replacing the corneal flap with the inlay sealed there between, such that the contour of the corneal surface is modified, the improvement comprising:
a) using an inlay which is biconvex in structure, optically clear and comprised of a flexible, biocompatible material having an index of refraction substantially the same as corneal tissue,
b) the inlay having a first and second surface, the first surfaces and the second surface being joined at a periphery of the inlay, the first and second surfaces having identical curvatures in an outer portion thereof and a centrally located portion on the first surface and the second surfaces, the centrally located portion having identical curvatures which are smaller in radius than the curvature of the outer portion of first and second surface, the inlay being no greater than 100 microns at its thickest point.
